(19) United States
(12) Patent Application Publication
(10) Pub. No.: US 2019/0281210 A1
(43) Pub. Date: Sep. 12, 2019

(54) Tool For Use With Image Capturing Device For Capturing Quality Image and Method Thereof

(71) Applicant: The Procter & Gamble Company, Cincinnati, OH (US)

(72) Inventors: Kukizo MIYAMOTO, Ashiya (JP); Tatsuya OMOTEZAKO, Nishinomiya (JP); Saumya BAREJA, Singapore (SG)

(21) Appl. No.: 16/294,991

(22) Filed: Mar. 7, 2019

Related U.S. Application Data
(60) Provisional application No. 62/640,034, filed on Mar. 8, 2018.

(51) Int. Cl. H04N 5/232; H04N 5/225; G06K 9/00

(52) U.S. Cl. CPC H04N 5/23219; H04N 5/2256; A61B 5/441; H04N 5/2254; G06K 9/00255

(57) ABSTRACT

A tool for use with an image capturing device for enabling capture of an image for analysis and/or evaluation. The tool includes a first end and a second end being connected by at least one wall. The first end is configured to be positioned over a surface enclosing a target facial area of a user for image capture thereof, and the second end is configured to receive the image capturing device. The second end includes an inner surface facing the first end and an outer surface. The tool may also include a reflective device located at the inner surface of the second end for aligning the position of the first end over the surface such that the target facial area is enclosed for image capture thereof.

### FIELD OF THE INVENTION

The present invention relates to a device or tool for use with an image capturing device for enabling capture of quality images and a method thereof, and more particularly, to a device or tool for use with an image capturing device for enabling users to capture quality facial images on their own in a consistent manner and that the captured images are reproducible and suitable for analysis and/or evaluation.

### BACKGROUND OF THE INVENTION

Skin counselling or facial skin counselling typically involves the use of skin imaging systems or tools for analysis and/or evaluation of a user's facial skin based on one or more captured facial images of the user. Skin imaging systems or tools are very useful for detecting, tracking and/or monitoring skin conditions and skin changes. Such skin imaging systems or tools typically have built-in image capturing capabilities.

The quality of a captured facial image plays a critical role in skin imaging as it may affect the accuracy of evaluation. Facial images should be captured in a consistent manner and reproducible. For example, to accurately monitor skin changes of a target facial area over time, it is important that at each time an image of the target facial area is captured, say once a week over a period of one month, factors such as the face position over which a skin imaging system or tool is placed for image capture, ambient lighting conditions and focal distance should be kept as consistent as possible so that the resulting image is captured in a consistent manner and reproducible for analysis and/or evaluation.

Current skin imaging systems or tools used for facial skin counselling that can capture quality images that are consistent and reproducible are typically found in skin clinics or beauty counters and are operated by dermatologists, skin therapists or beauty consultants. Such skin imaging systems are expensive and difficult to deploy with scale. Furthermore, with an increasing number of consumers who prefer e-commerce over in-store when purchasing skin or facial products, it would be difficult for such consumers to perform self-skin counselling at home without any tool or device to assist them in capturing images of desired facial areas in a consistent manner and that are reproducible and suitable for further analysis and/or evaluation. Without such a tool or device, images may be captured under different lighting conditions that may not be reproducible and suitable for analysis and/or evaluation. The problem may be exacerbated when consumers attempt to capture multiple images of the same desired facial area over time as it would be difficult to keep factors such as focal distance and position of a camera with respect to the desired facial area consistent each time an image is captured.

There is therefore a need for a low-cost device or tool that is suitable for use with an image capturing device for enabling users to capture quality facial images on their own in a consistent manner and that the captured images are reproducible and suitable for analysis and/or evaluation.

### SUMMARY OF THE INVENTION

The present invention relates to a tool for use with an image capturing device for enabling capture of a quality image. The tool comprises a first end and a second end being connected by at least one wall, and the distance between the first end and the second end is predetermined. The first end is configured to be positioned over a surface enclosing a target facial area of a user for image capture thereof, and the second end is configured to receive the image capturing device. The second end comprises an inner surface facing the first end and an outer surface. The tool also comprises a reflective device located at the inner surface of the second end at a predetermined location for aligning the position of the first end over the surface such that the target facial area is enclosed for image capture thereof by enabling the user to locate the reflection of a predetermined object in the reflective device. When the user locates the reflection of the predetermined object in the mirror, a quality image of the target facial area may be captured by the image capturing device.

These and other aspects of the present invention will become more apparent upon reading the following detailed description of the invention.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate various embodiments described herein, and together with the description serve to explain the principles and operations of the claimed subject matter.

The present invention will now be described, by way of illustrative example only, with reference to the accompanying drawings, of which:

FIG. 1 is a perspective view of a tool for use with an image capturing device for enabling capture of a quality image of a target facial area in accordance with the present invention;

FIG. 2 is a side view of the tool of FIG. 1 when in use by a user;

FIG. 3A is a plan view of a product carton that is unfolded in which a design portion configured to provide a tool is printed on a surface of the product carton in accordance with the present invention;

FIGS. 3B to 3E are plan views of four external panels of the product carton of FIG. 3A;

FIGS. 3F and 3G are perspective views of the product carton of FIG. 3A when folded;

FIG. 3H is a plan view of the design portion removed from the product carton of FIG. 3A;

FIG. 4A is a plan view of a product carton that is unfolded in which a design portion configured to provide a tool is printed on a surface of the product carton in accordance with the present invention;

FIGS. 4B to 4E are plan views of four external panels of the product carton of FIG. 4A;

FIGS. 4F and 4G are perspective views of the product carton of FIG. 4A when folded;

FIG. 4H is a plan view of the design portion removed from the product carton of FIG. 4A;

FIG. 5A is a perspective view of a tool in accordance with the present invention in which the target facial area is the cheek area;

FIG. 5B is a front view of a portion of the tool of FIG. 5A;

FIG. 6A is a perspective view of a tool in accordance with the present invention in which the target facial area is the nose area;

FIG. 6B is a front view of a portion of the tool of FIG. 6A;

FIG. 7A is a perspective view of a tool in accordance with the present invention in which the target facial area is the forehead area;

FIG. 7B is a front view of a portion of the tool of FIG. 7A;

FIG. 8A is a perspective view of a tool in accordance with the present invention in which the target facial area is one or more of the following areas: mouth area, chin area and one or more teeth area;

FIG. 8B is a front view of a portion of the tool of FIG. 8A;

FIG. 9A is a perspective view of a tool in accordance with the present invention in which the target facial area is the periorbital area;

FIG. 9B is a front view of a portion of the tool of FIG. 9A;

FIG. 10A is a perspective view of a tool in accordance with the present invention in which the target facial area is the periorbital area;

FIG. 10B is a front view of a portion of the tool of FIG. 10A;

FIG. 11A is a perspective view of a tool in accordance with the present invention in which the target facial area is the entire face area; and

FIG. 11B is a front view of a portion of the tool of FIG. 11A.

### DETAILED DESCRIPTION OF THE INVENTION

Features and benefits of the various embodiments of the present invention will become apparent from the following description, which includes examples of specific embodiments intended to give a broad representation of the invention. Various modifications will be apparent to those skilled in the art from this description and from practice of the invention. The scope of the present invention is not intended to be limited to the particular forms disclosed and the invention covers all modifications, equivalents, and alternatives falling within the spirit and scope of the invention as defined by the claims.

As used herein, the term “quality image” refers to an image which is captured in a consistent, reproducible manner and is suitable for analysis and/or evaluation. To capture an image in a consistent manner, factors such as, the face position over which a tool for use with an image capturing device is placed for image capture, ambient lighting and focal distance, should be kept as consistent as possible. In instances where multiple images of a desired or target facial area are required, for example, to monitor skin changes of a target facial area over time, for each of the multiple images to be regarded as a “quality image”, each of these images should be captured in a consistent, reproducible manner and is suitable for analysis and/or evaluation.

As used herein, the term “facial area” refers to a user's entire face or a portion of a user's face, including, but not limited to one or more of the following areas: cheek, nose, forehead, mouth, chin, one or more teeth, periorbital area, and entire face.

As used herein, the term “image capturing device” refers to a device or instrument that can capture and/or record images (e.g., still pictures or videos). The device may be part of a mobile device or a smart device, which includes a mobile phone, a smart phone, a tablet, a laptop, a watch, a personal digital assistant, or may be part of a personal computer, or may be a standalone camera such as a handheld camera. The device may also include a built-in light source (e.g., a flash) for emitting light.

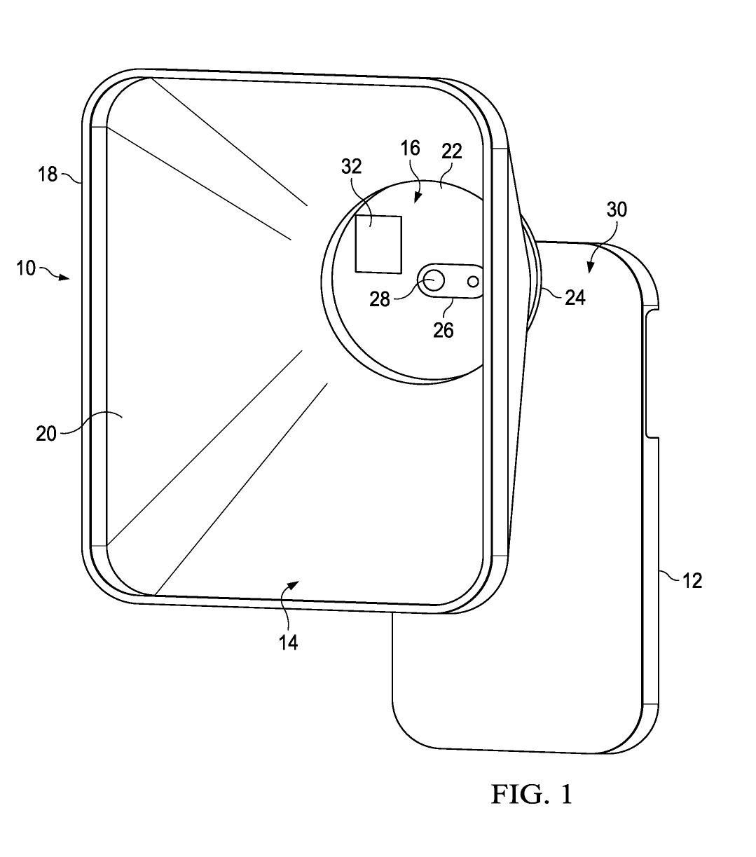

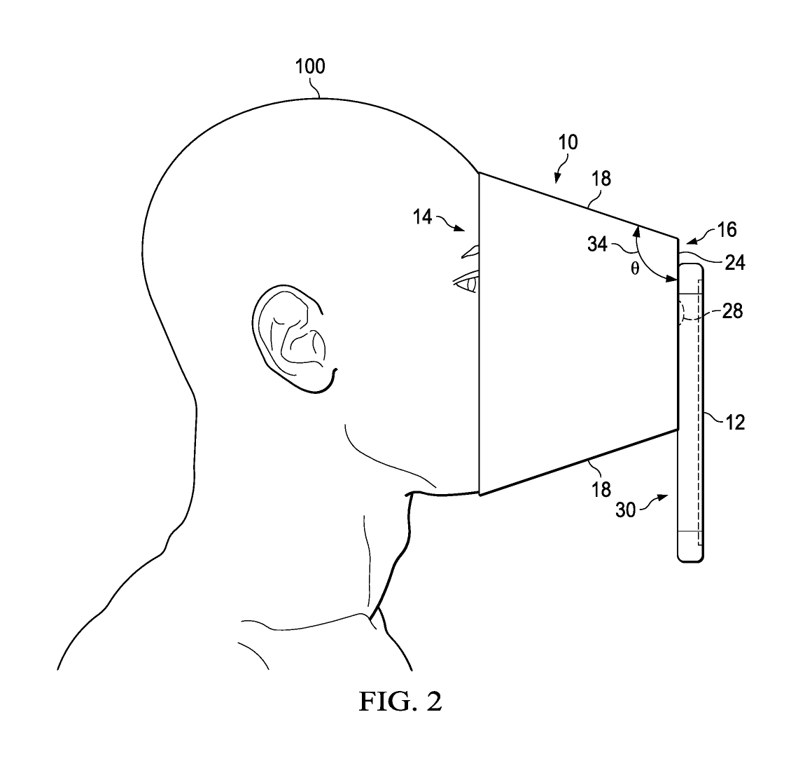

FIG. 1 is a perspective view of a tool 10 for use with an imaging capturing device 12 for enabling capture of a quality image, and FIG. 2 is a side view of the tool 10 when in use by a user 100. As illustrated, the tool 10 comprises a first end 14 and a second end 16 connected by walls 18 to form a hollow structure 20, and the distance between the first end 14 and the second end 16 is predetermined. The first end 14 is configured to be positioned over a surface (e.g., a portion of skin of a facial area) enclosing a target facial area of the user 100 for image capture thereof. The target facial area for image capture may include, but is not limited to any one of the following facial areas: cheek area, nose area, forehead area, mouth area, chin area, one or more teeth, periorbital area and entire face area.

The first end 14 may be generally rectangular and may be in the form of an open bottom end as shown in FIG. 1 and FIG. 2. Preferably, the surface area of the first end 14 is in the range of about 1,000 mm² to about 33,000 mm². While the first end 14 is illustrated as having a generally rectangular shape, it should be understood that it can take on other shapes (e.g., circular, oblong, square, triangular, or any other polygonal shape, etc.).

Preferably, the predetermined distance between the first end 14 and the second end 16 is in the range of about 80 mm to about 600 mm. This preferred range enables a clear focus of the image for capture and at the same time enables the user 100 to comfortably capture a quality facial image or images on the user's own without having to overstretch the user's arm when using the tool 10. The minimum distance between the first end 14 and the second end 16 may correspond to the minimum focusing distance of the image capturing device 12, and it should be understood that the focusing distance may be less than 80 mm depending on the specifications of the image capturing device 12. It should also be understood that the maximum distance between the first end 14 and the second 16 may be greater than 600 mm as long as it is still feasible for the user 100 to use to capture a quality image on the user's own.

The second end 16 comprises an inner surface 22 facing the first end 14 and an outer surface 24, and is configured to receive and/or position the image capturing device 12. The second end 16 may be circular shown in FIG. 1. Preferably, the surface area of the second end 16 is in the range of about 1,000 mm² to about 33,000 mm². While the second end 16 is illustrated as having a circular shape, it should be understood that it can take on other shapes (e.g., oblong, square, triangular, rectangular or any other polygonal shape, etc.). The second end 16 comprises a window 26 for positioning of a lens 28 of the image capturing device 12 when a lens-side surface 30 of the image capturing device 12 is adjacent to the outer surface 24 of the second end 16.

The tool 10 further comprises a reflective device 32, such as a mirror or any reflective surface which reflects an image, located at the inner surface 22 of the second end 16 for aligning the position of the first end 14 over the surface such that the target facial area is enclosed by the hollow structure 20 for image capture thereof by enabling the user 100 to locate the reflection of a predetermined object, such as the user's eye, the user's eye brow, the user's one or more laugh lines or crow's feet in the periorbital area, or any suitable reference point in the periorbital area or facial area, in the reflective device 32. Preferably, the predetermined object is the user's eye. The location of the reflective device 32 at the inner surface 22 of the second 16 is predetermined. When the user 100 locates the reflection of the predetermined object in the reflective device 32, a quality image of the target facial area may be captured by the image capturing device 12. The image may be captured manually or automatically. Preferably, the tool 10 is used in conjunction with a software application or “app” which has a recognition feature for the predetermined object for enabling automatic image capture by the image capturing device 12 when the user 100 locates the reflection of the predetermined object in the reflective device 32.

For typical mobile phones and other handheld devices, the predetermined location of the reflective device 32 is typically at a distance of about 3 mm to about 106 mm with respect to the lens 28 of the image capturing device 12 when the lens-side surface 30 of the image capturing device 12 is adjacent to the outer surface 24 of the second end 16, although the predetermined location can be any suitable location for the desired result. The location of the reflective device 32 can be determined based on the choice of the predetermined object and desired facial area that is being observed. The distance between the reflective device 32 with respect to the lens 28 is measured from the center of the lens 28 to an edge of the reflective device 32 that is closest to the lens 28. The position of the reflective device 32 may be fixed at a specific spot or may be adjustable, such as by the user 100, provided the distance between the reflective device 32 with respect to the lens 28 is within the distance of about 3 mm to about 106 mm. The reflective surface area of the reflective device 32 is preferably in the range of about 80 mm² to about 650 mm². While the reflective device 32 is illustrated as having a rectangular shape, it should be understood that it can take on other shapes (e.g., circular, oblong, square, triangular, or any other polygonal shape, etc.).

The inner surface 22 of the second end 16 forms an angle 34 with the walls 18 and the angle 34 preferably ranges from about 80 degrees to about 135 degrees. More preferably, the angle 34 ranges from about 90 degrees to about 135 degrees. These ranges advantageously ensure that the surface enclosed by the first end 14 adequately covers the target facial area and prevent or minimize the walls 18 of the tool 10 from being captured as part of the image of the target facial area.

The tool 10 may further comprise a connector for facilitating the connection between the tool 10 and the image capturing device 12 such that the lens-side surface 30 of the image capturing device 12 is adjacent to the outer surface 24 of the second end 16. Examples of the connector include an adhesive, a hook and loop fastener, a magnetic fastener, a clip-on fastener, an elastic fastener, a suction cup fastener and the like. In some embodiments, the image capturing device 12 may be connected to the tool 10 by manually holding in place the lens-side surface 30 of the image capturing device 12 against the outer surface 24 of the second end 16 such that the lens-side surface 30 is adjacent to the outer surface 24 with the lens 28 aligned within the window 26.

The tool 10 may further comprise a light source located at the inner surface 24 of the second end 16. Preferably, the light source is a LED light source, but any suitable light source can be used. The image capturing device 12 may comprise a built-in light source such as a LED light source or an external light source can be used. The image capturing device 12 may be any device able to capture suitable images for the desired use, including, but not limited to a part of a mobile device or smart device, preferably a mobile phone, a smart phone, a tablet, a laptop, a watch, or a personal digital assistant, or may be part of a personal computer, or may be a handheld camera.

The tool 10 may be made from any suitable material. For example, the tool 10 may be made of one or more materials which prevent or reduce ambient or environmental light from penetrating through the tool 10 to create a controlled lighting space avoiding ambient or environmental lighting conditions within the hollow structure 20 of the tool 10. The walls 18 of the tool 10 are preferably opaque and may be made from materials such as paper, paperboard, polystyrene, polystyrene terephthalate, polypropylene, and silicon rubber. The tool 10 may also be disposable or reusable. The tool 10 may also be foldable into a flat plane when it is not in use.

A cross section of the tool 10 may have a substantially rectangular shape. It should be understood, though, that the cross section of the tool 10 can take on other shapes (e.g., circular, quadrangular pyramid, quadrangular prism, or elliptical cone, etc.).

Advantageously, the tool 10 can be used to align the lens 28 of the image capturing device 12 with the same target facial area at the same distance throughout multiple uses. Thus, the tool 10 enables the user 100 to capture images of the same target facial area under consistent light conditions, consistent focal distance and consistent position over which the tool 10 is placed for image capture, and such images are reproducible and suitable for analysis and/or evaluation which allows for accurate evaluation of the target facial area over time.

There is described hereinafter a method for capturing a quality image using the image capturing device 12 and the tool 10 in accordance with the present invention. The method comprises locating the second end 16 of the tool 10 to a lens-side surface 30 of the image capturing device 12 by a connector such that the lens-side surface 30 of the image capturing device 12 is adjacent to the outer surface 24 of the second end 16. The method further comprises positioning the first end 14 of the tool 10 over a surface enclosing a target facial area of the user 100 for image capture thereof. The method also comprises aligning the position of the first end 14 such that the target facial area is enclosed for image capture thereof by enabling the user 100 to locate the reflection of a predetermined object, such as the user's eye, the user's eye brow, the user's one or more laugh lines or crow's feet in the periorbital area, or any suitable reference point in the periorbital area or facial area, in the reflective device 32, and capturing, using the image capturing device 12, a quality image of the target facial area when the user 100 locates the reflection of the predetermined object in the reflective device 32. Preferably, the predetermined object is the user's eye. The method further comprises using the tool 10 in conjunction with a software application or “app” having a recognition feature for the predetermined object for enabling automatic image capture by the image capturing device 12 when the user 100 locates the reflection of the predetermined object in the reflective device 32.

The tool 10 may comprise a carton or a portion of a carton of a product, such as, for example, a skin care product or an oral care product. The design of the tool 10 may be printed on a surface of a carton (e.g. the outer package or box of a product) or may be cut or perforated into the material of the carton. A product, such as a skin car product or an oral care product, may be housed within an interior of the carton. The user 100 can be provided with instructions for modifying the carton (e.g. by punching out, cutting, folding and/or gluing) into the tool 10.

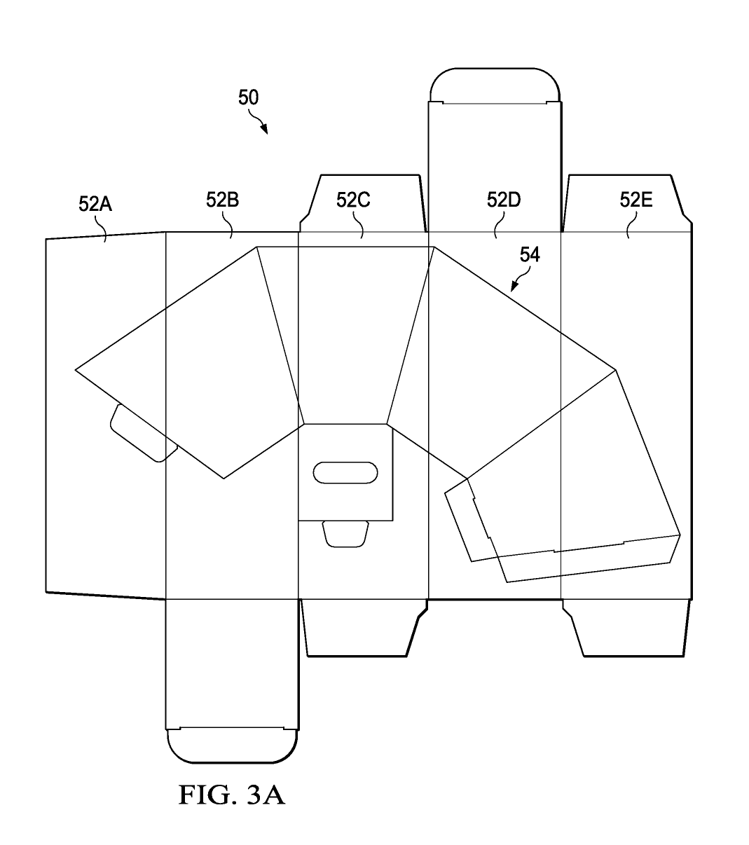

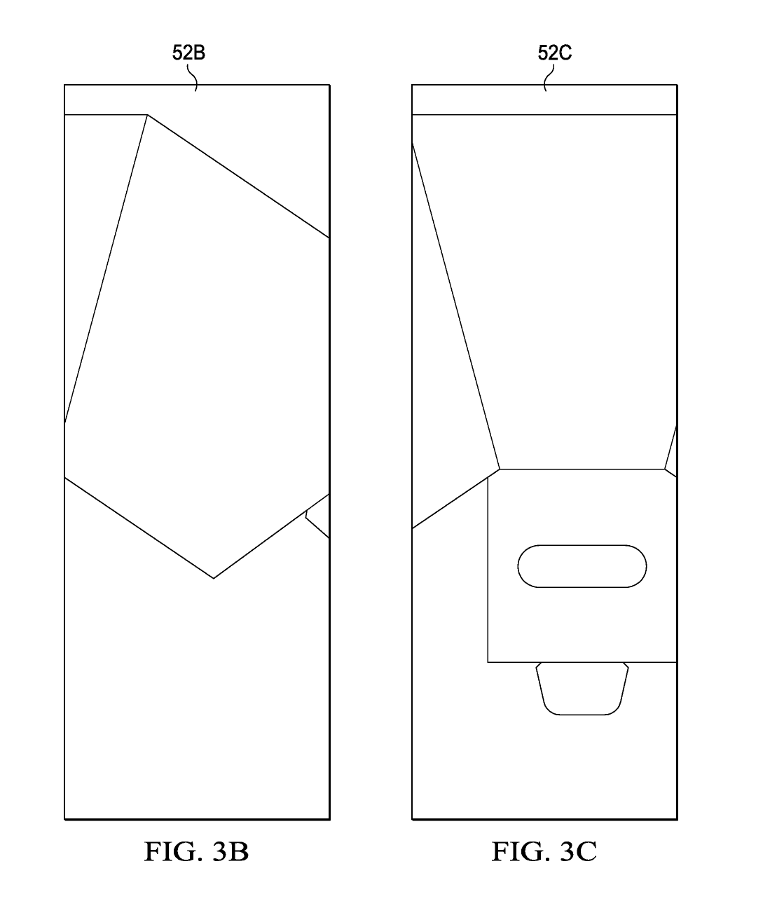

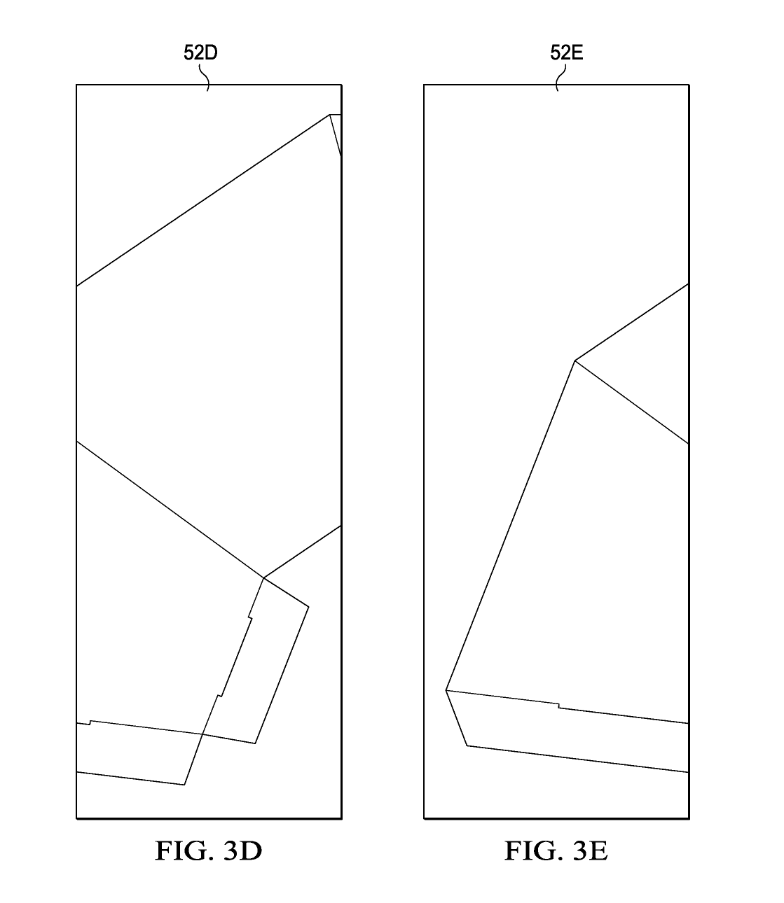

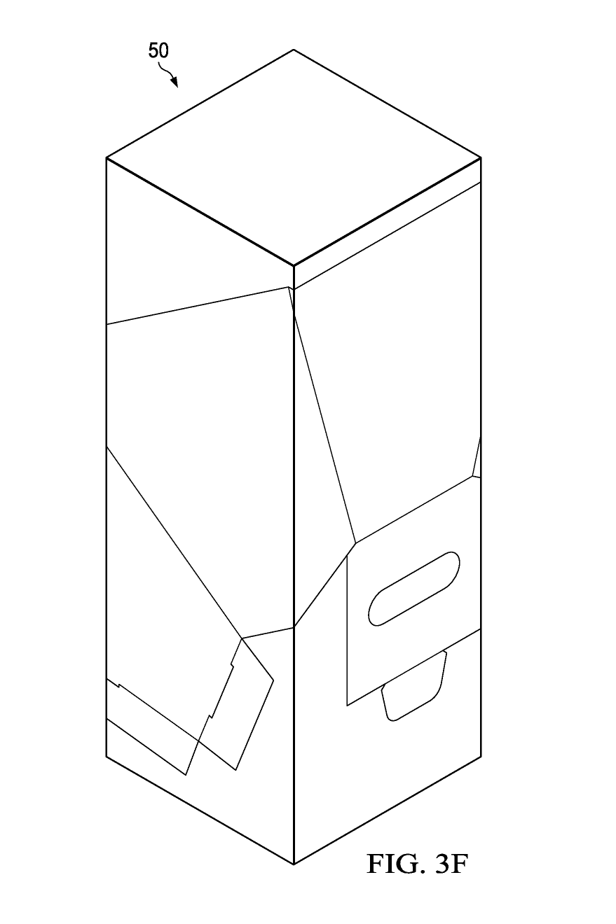

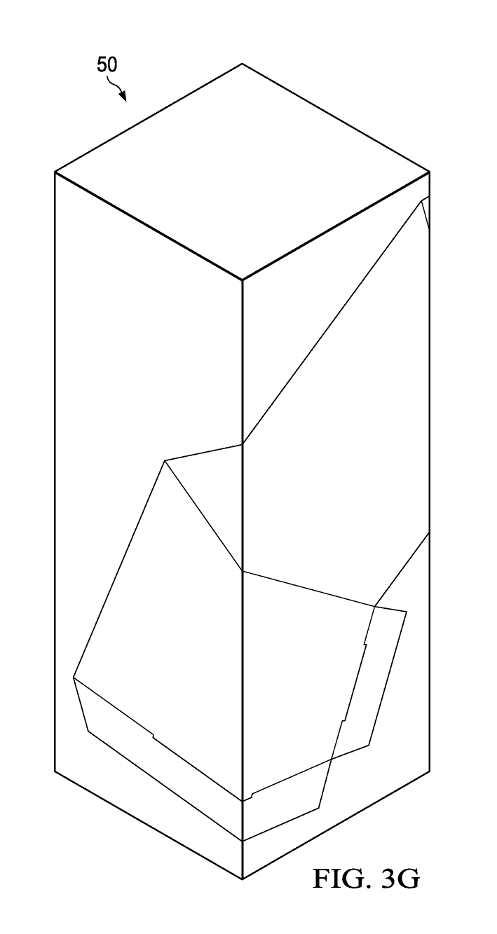

FIG. 3A illustrates a plan view of a product carton 50 that is unfolded (i.e., a carton blank) in accordance with the present invention. The product carton 50 comprises an internal surface and an external surface, in which at least one of the surfaces comprises a design portion 54 configured to provide the tool 10. The design portion 54 may be printed on at least one of the surfaces. In this example, the design portion 54 is printed on external panels 52A to 52E, as illustrated in FIG. 3A. While the design portion 54 is illustrated as being printed on the product carton 50, it should be understood that the design portion 54 can be cut or perforated into the material of the product carton 50.

When the product carton 50 is folded, the panels 52B to 52E are visible to the user 100 and the panel 52A is not visible to the user 100, as illustrated in FIGS. 3B to 3G. In another example, the design portion 54 may be printed on the reverse sides of the external panels 52A to 52E (i.e., on the internal surface of the product carton 50), and the design portion 54 is not visible to the user when the product carton 50 is folded. In another example, the design portion 54 may be printed on one or more of the external panels 52A to 52E and on one or more of the reverse sides of the external panels 52A to 52E, such that a part of the design portion 54 is visible to the user 100 and another part of the design portion 54 is not visible to the user 100 when the product carton 50 is folded.

The design portion 54 may be removed from the product carton 50 when the product carton 50 is unfolded or folded. As illustrated in FIG. 3H, when the design portion 54 is removed from the product carton 50, the removed design portion 54 may be assembled to form the tool 10. In this example, the tool 10 takes on the shape of a quadrangular prism when assembled. The design portion 54 may comprise the reflective device 32 which is located at the inner surface 22 of the second end 16 of the tool 10 when the tool 10 is assembled. In another example, the reflective device 32 may not be part of the design portion 54 and may be provided separately.

Instructions for assembling the tool 10 from the removed design portion 54 may be provided with the product carton 50, such as, instructions for modifying the product carton 50, by punching out, cutting, folding and/or gluing, into the tool 10.

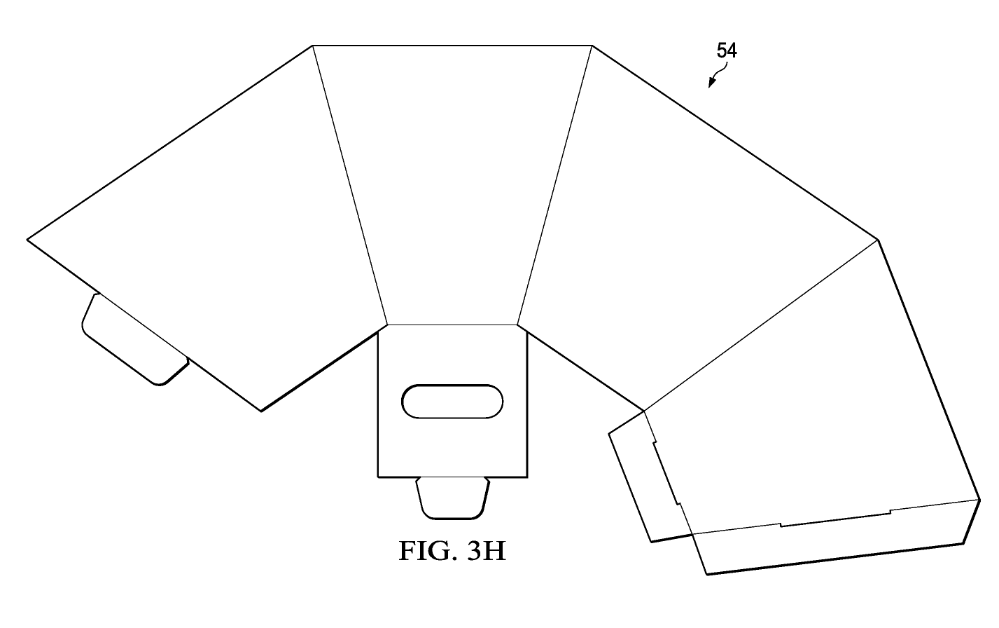

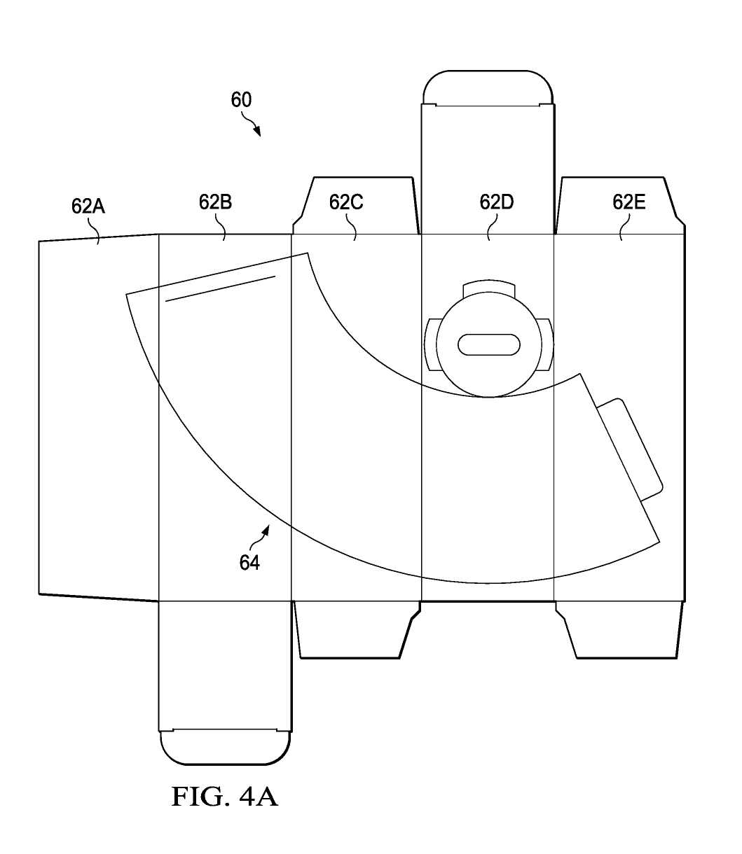

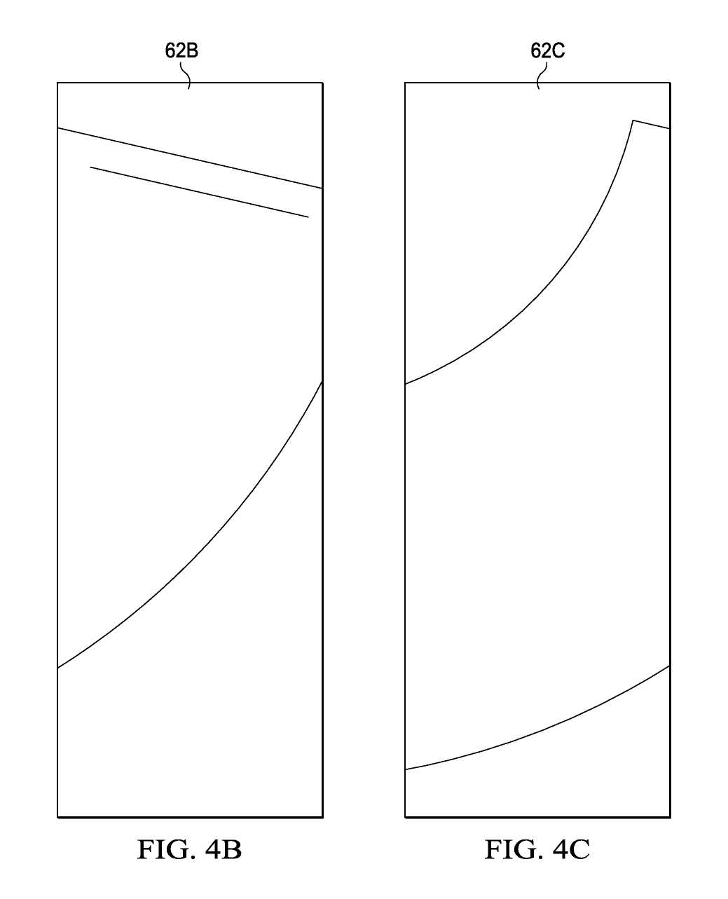

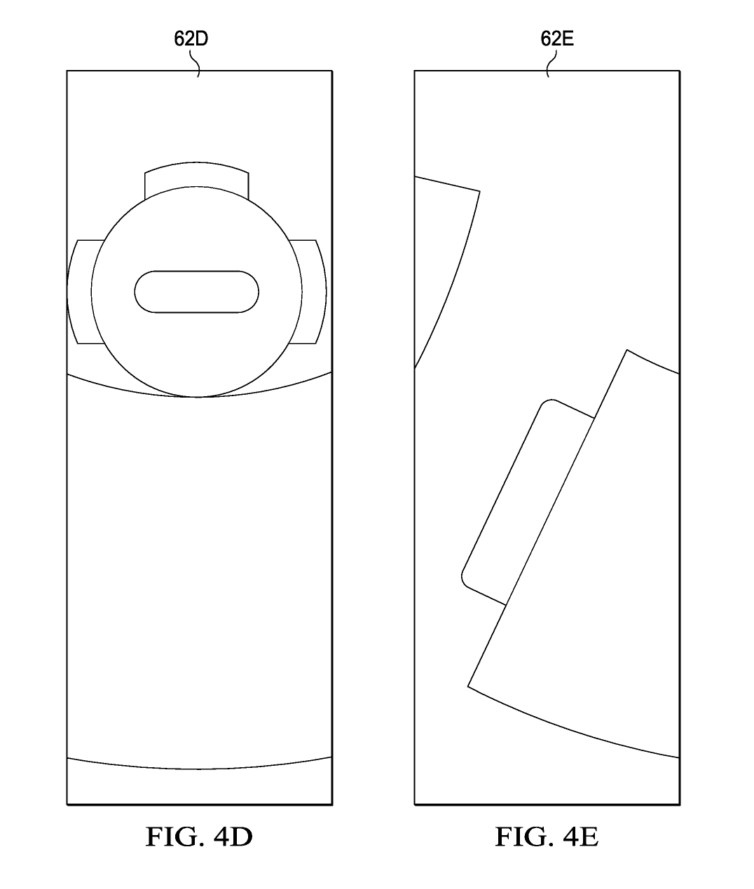

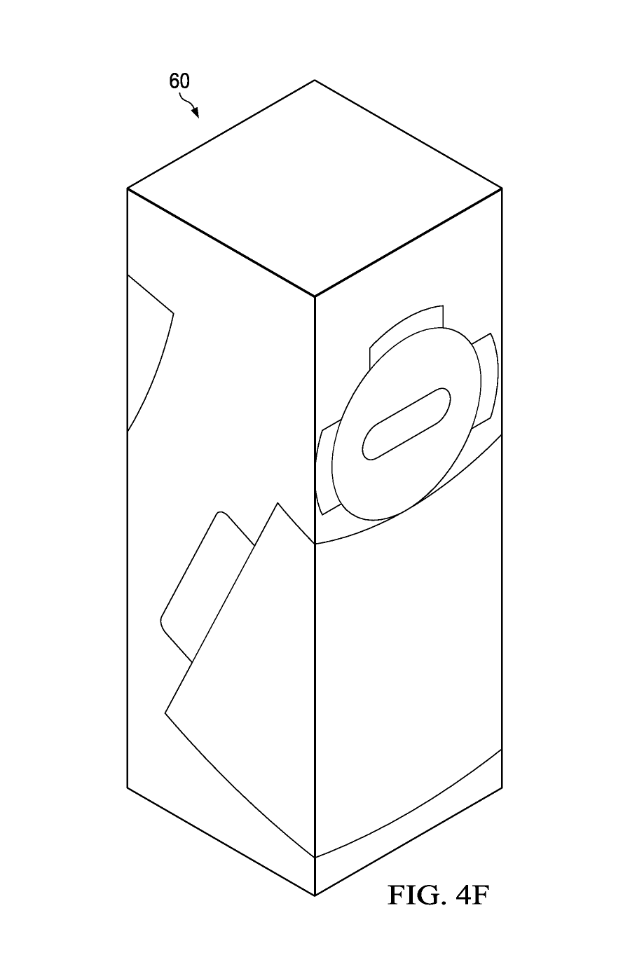

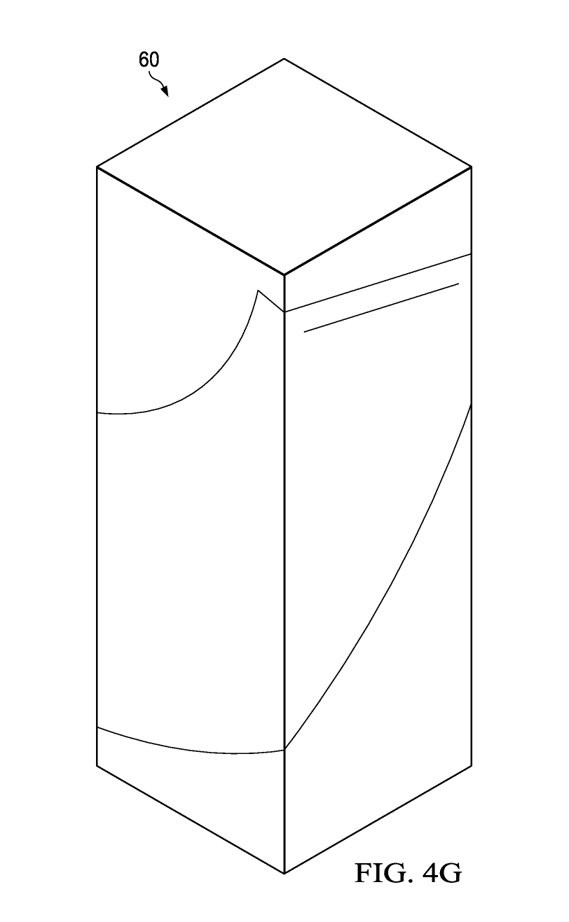

FIG. 4A illustrates a plan view of a product carton 60 that is unfolded (i.e., a carton blank) in accordance with the present invention. The product carton 60 comprises an internal surface and an external surface, in which at least one of the surfaces comprises a design portion 64 configured to provide the tool 10. The design portion 64 may be printed on at least one of the surfaces. In this example, the design portion 64 is printed on external panels 62A to 62E, as illustrated in FIG. 4A. While the design portion 64 is illustrated as being printed on the product carton 60, it should be understood that the design portion 64 can be cut or perforated into the material of the product carton 60.

When the product carton 60 is folded, the panels 62B to 62E are visible to the user 100 and the panel 62A is not visible to the user 100, as illustrated in FIGS. 4B to 4G. In another example, the design portion 64 may be printed on the reverse sides of the external panels 62A to 62E (i.e., on the internal surface of the product carton 60), and the design portion 64 is not visible to the user when the product carton 60 is folded. In another example, the design portion 64 may be printed on one or more of the external panels 62A to 62E and on one or more of the reverse sides of the external panels 62A to 62E, such that a part of the design portion 64 is visible to the user 100 and another part of the design portion 64 is not visible to the user 100 when the product carton 60 is folded.

The design portion 64 may be removed from the product carton 60 when the product carton 60 is unfolded or folded. As illustrated in FIG. 4H, when the design portion 64 is removed from the product carton 60, the removed design portion 64 may be assembled to form the tool 10. In this example, the tool 10 takes on the shape of a circular prism when assembled. The design portion 64 may comprise the reflective device 32 which is located at the inner surface 22 of the second end 16 of the tool 10 when the tool 10 is assembled. In another example, the reflective device 32 may not be part of the design portion 64 and may be provided separately.

Instructions for assembling the tool 10 from the removed design portion 64 may be provided with the product carton 60, such as, instructions for modifying the product carton 60, by punching out, cutting, folding and/or gluing, into the tool 10.

### EXAMPLES

### Cheek Area

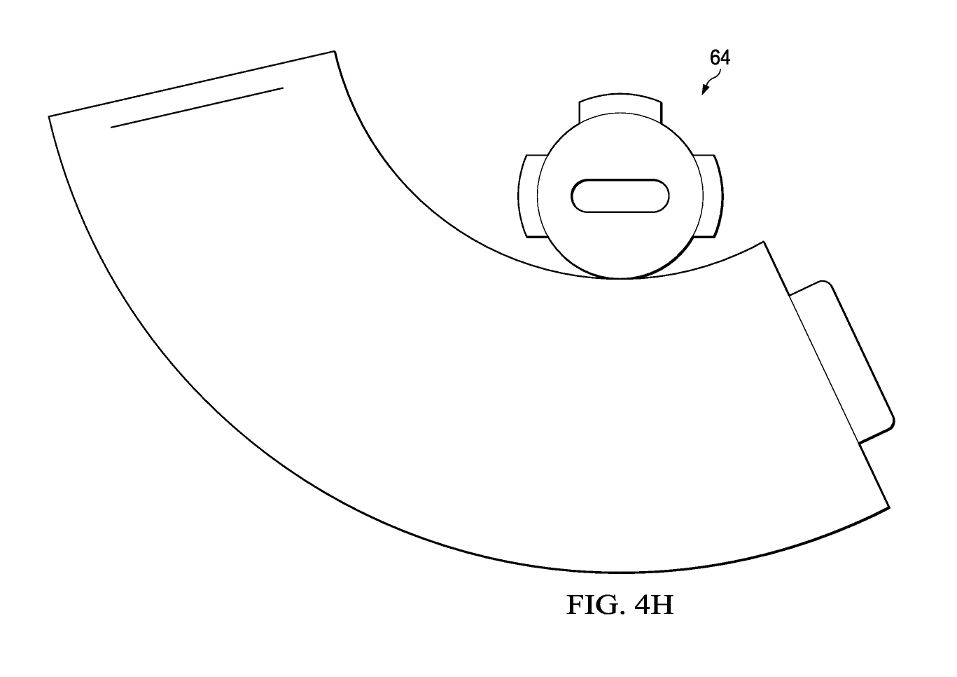

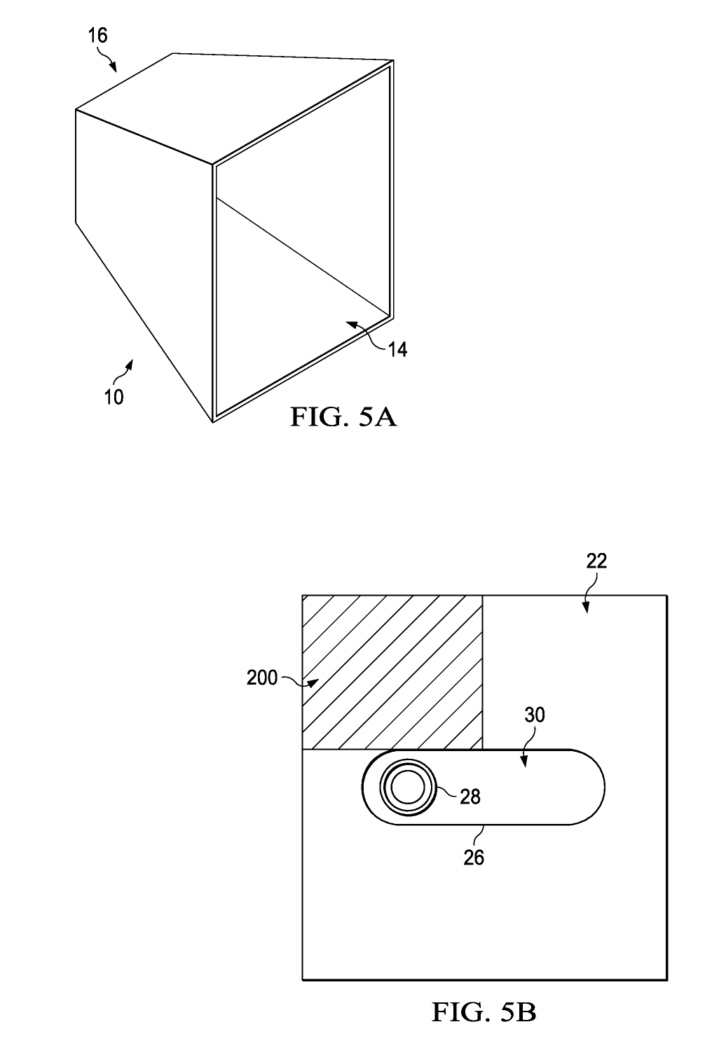

FIG. 5A is a perspective view of the tool 10 in accordance with the present invention in which the target facial area for image capture is the cheek area. In this example, the shape of the first end 14 is rectangular, and the length and breadth of the first end 14 is about 125 mm and about 101 mm, respectively. The shape of the second end 16 of the tool 10 is substantially a square, and the length and breadth of the inner surface 22 of the second end 16 is about 55 mm and about 54 mm, respectively, as illustrated in FIG. 5B. The predetermined distance between the first end 14 and the second end 16 is about 100 mm.

The predetermined location of the reflective device 32 at the inner surface 22 is defined by a shaded portion 200 of the inner surface 22 as illustrated in FIG. 5B. The length and breadth of the shaded portion 200 is about 30 mm and about 21.5 mm, respectively. The reflective device 32 may be placed anywhere on the shaded portion 200. In the example shown, the reflective device 32 is shaped in the form of a square measuring 13 mm by 13 mm.

### Nose Area

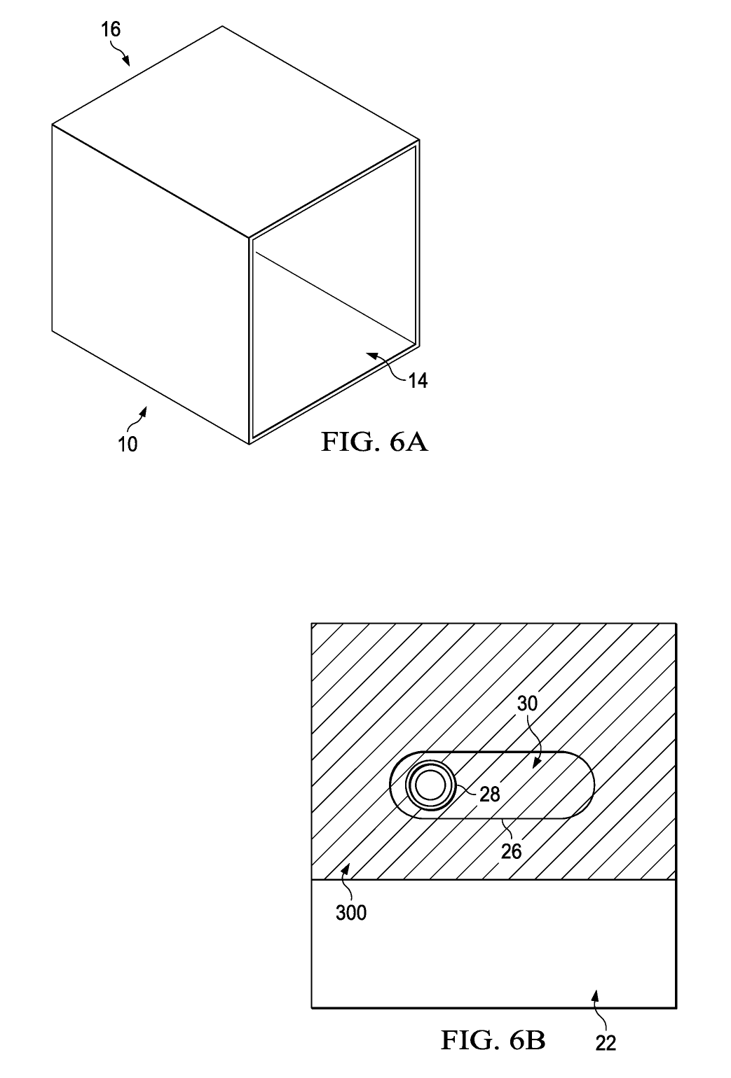

FIG. 6A is a perspective view of another example of the tool 10 of the present invention in which the target facial area for image capture is the nose area. In this example, the shape of the first end 14 is rectangular, and the length and breadth of the first end 14 is about 100 mm and about 95 mm, respectively. The shape of the second end 16 of the tool 10 is rectangular, and the length and breadth of the inner surface 22 of the second end 16 is about 100 mm and about 95 mm, respectively, as illustrated in FIG. 6B. The predetermined distance between the first end 14 and the second end 16 is about 110 mm.

The predetermined location of the reflective device 32 at the inner surface 22 is defined by a shaded portion 300 of the inner surface 22 as illustrated in FIG. 6B. The length and breadth of the shaded portion 200 is about 95 mm and about 70 mm, respectively. The reflective device 32 may be placed anywhere on the shaded portion 300. In the example shown, the reflective device 32 is shaped in the form of a square measuring 13 mm by 13 mm.

### Forehead Area

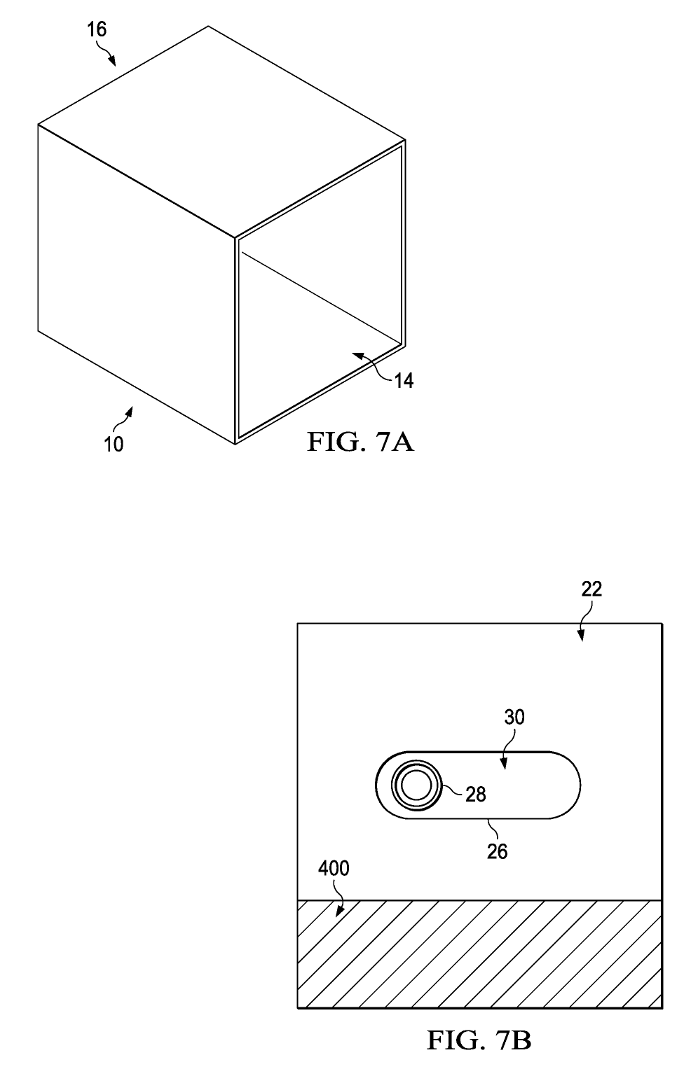

FIG. 7A is a perspective view of the tool 10 in accordance with another example of the present invention in which the target facial area for image capture is the forehead area. In this embodiment, the shape of the first end 14 is rectangular, and the length and breadth of the first end 14 is about 100 mm and about 95 mm, respectively. The shape of the second end 16 of the tool 10 is rectangular, and the length and breadth of the inner surface 22 of the second end 16 is about 100 mm and about 95 mm, respectively, as illustrated in FIG. 7B. The predetermined distance between the first end 14 and the second end 16 is about 110 mm.

The predetermined location of the reflective device 32 at the inner surface 22 is defined by a shaded portion 400 of the inner surface 22 as illustrated in FIG. 7B. The length and breadth of the shaded portion 400 is about 95 mm and about 35 mm, respectively. The reflective device 32 may be placed anywhere on the shaded portion 400. In the example shown, the reflective device 32 is shaped in the form of a square measuring 13 mm by 13 mm

### Mouth Area, Chin Area, One or More Teeth Area

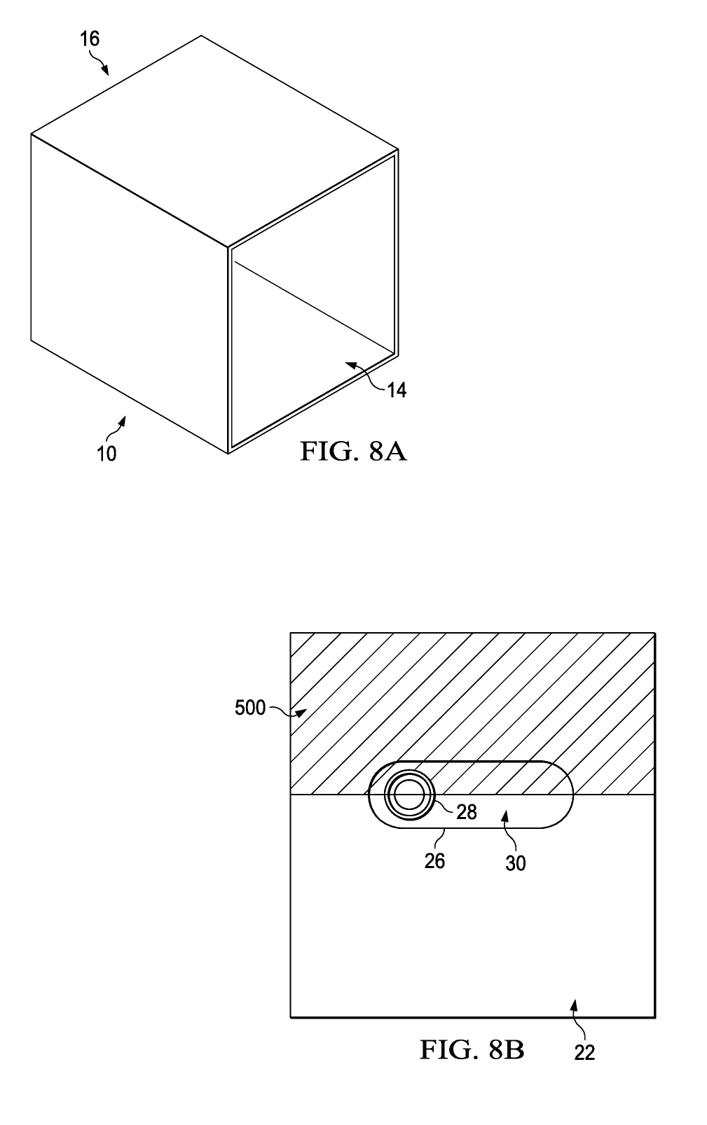

FIG. 8A is a perspective view of the tool 10 in accordance with another example of the present invention in which the target facial area for image capture is one or more of the following facial areas: mouth area, chin area and one or more teeth area. In this example, the shape of the first end 14 is rectangular, and the length and breadth of the first end 14 is about 100 mm and about 95 mm, respectively. The shape of the second end 16 of the tool 10 is rectangular, and the length and breadth of the inner surface 22 of the second end 16 is about 100 mm and about 95 mm, respectively, as illustrated in FIG. 8B. The predetermined distance between the first end 14 and the second end 16 is about 100 mm.

The predetermined location of the reflective device 32 at the inner surface 22 is defined by a shaded portion 500 of the inner surface 22 as illustrated in FIG. 8B. The length and breadth of the shaded portion 500 is about 95 mm and about 40 mm, respectively. The reflective device 32 may be placed anywhere on the shaded portion 500. In the example shown, the reflective device 32 is shaped in the form of a square measuring 13 mm by 13 mm.

### Periorbital Area

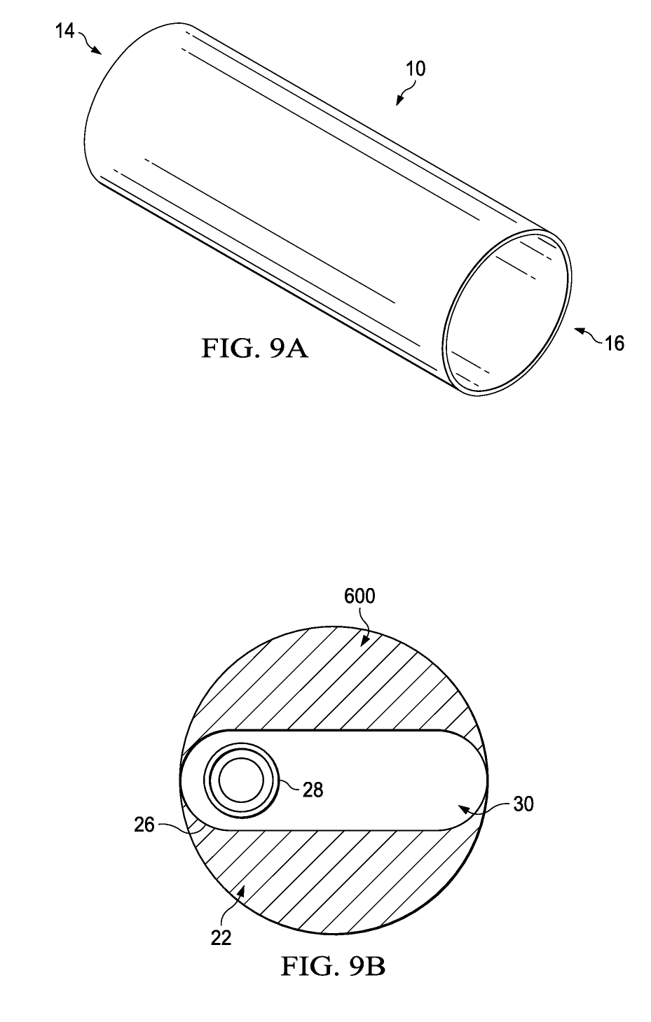

FIG. 9A is a perspective view of the tool 10 in accordance with another example of the present invention in which the target facial area for image capture is the periorbital area. In this example, the shape of the first end 14 is circular, and the diameter of the first end 14 is about 36 mm. The shape of the second end 16 of the tool 10 is also circular, and the diameter of the inner surface 22 of the second end 16 is about 36 mm, as illustrated in FIG. 9B. The predetermined distance between the first end 14 and the second end 16 is about 100 mm.

The predetermined location of the reflective device 32 at the inner surface 22 is defined by a shaded portion 600 of the inner surface 22 as illustrated in FIG. 9B. The diameter of the shaded portion 600 is about 36 mm. The reflective device 32 may be placed anywhere on the shaded portion 600. In the example shown, the reflective device 32 is shaped in the form of a square measuring 13 mm by 13 mm.

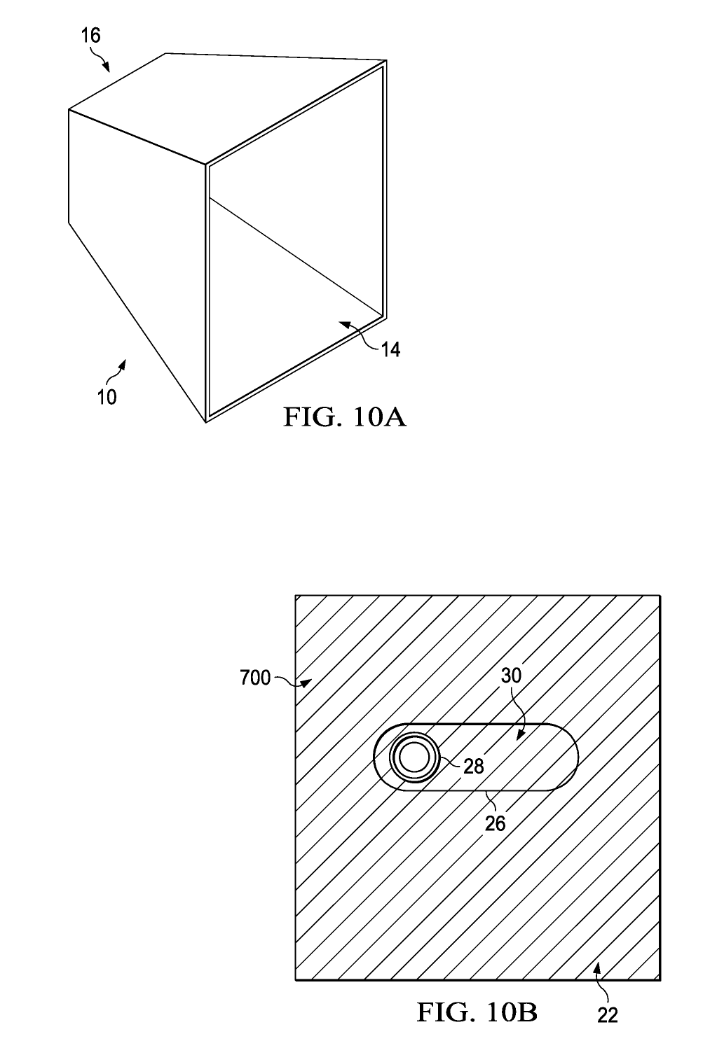

FIG. 10A is a perspective view of the tool 10 in accordance with another example of the present invention in which the target facial area for image capture is the periorbital area. In this example, the shape of the first end 14 is rectangular, and the length and breadth of the first end 14 is about 125 mm and 101 mm, respectively. The shape of the second end 16 of the tool 10 is substantially a square, and the length and breadth of the inner surface 22 of the second end 16 is about 55 mm and about 54 mm, respectively, as illustrated in FIG. 10B. The predetermined distance between the first end 14 and the second end 16 is about 100 mm.

The predetermined location of the reflective device 32 at the inner surface 22 is defined by a shaded portion 700 of the inner surface 22 as illustrated in FIG. 10B. The length and breadth of the shaded portion 700 is about 55 mm and about 54 mm, respectively. The reflective device 32 may be placed anywhere on the shaded portion 700. In the example shown, the reflective device 32 is shaped in the form of a square measuring 13 mm by 13 mm.

### Entire Face Area

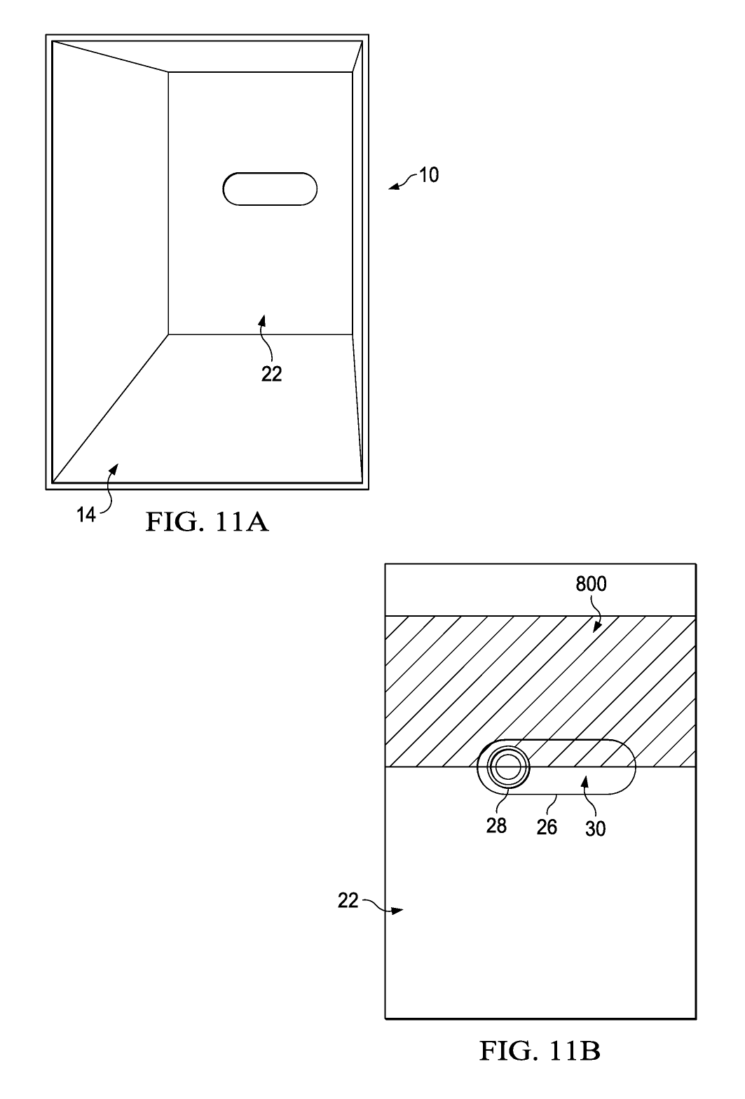

FIG. 11A is a perspective view of the tool 10 in accordance with another example of the present invention in which the target facial area for image capture is the entire face area. In this example, the shape of the first end 14 is rectangular, and the length and breadth of the first end 14 is about 220 mm and about 150 mm, respectively. The shape of the second end 16 of the tool 10 is rectangular, and the length and breadth of the inner surface 22 of the second end 16 is about 220 mm and about 150 mm, respectively, as illustrated in FIG. 11B. The predetermined distance between the first end 14 and the second end 16 is about 110 mm.

The predetermined location of the reflective device 32 at the inner surface 22 is defined by a shaded portion 800 of the inner surface 22 as illustrated in FIG. 11B. The length and breadth of the shaded portion 800 is about 150 mm and about 115 mm, respectively. The reflective device 32 may be placed anywhere on the shaded portion 800. In the example shown, the reflective device 32 is shaped in the form of a square measuring 13 mm by 13 mm.

It will be understood that reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm ” All numeric ranges described herein are inclusive of narrower ranges; delineated upper and lower range limits are interchangeable to create further ranges not explicitly delineated. Embodiments described herein can comprise, consist essentially of, or consist of, the essential components as well as optional pieces described herein. As used in the description and the appended claims, the singular forms “a,” “an,” and “the” are intended to include the plural forms as well, unless the context clearly indicates otherwise.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tool for use with an image capturing device for enabling capture of a quality image, comprising:
a first end and a second end being connected by at least one wall, the first end configured to be positioned over a surface enclosing a target facial area of a user for image capture thereof, the second end configured to receive the image capturing device, the second end comprises an inner surface facing the first end and an outer surface; and
a reflective device located at the inner surface of the second end for aligning the position of the first end over the surface such that the target facial area is enclosed for image capture thereof by enabling the user to locate the reflection of a predetermined object in the reflective device,
wherein the distance between the first end and the second end is predetermined and the location of the reflective device at the inner surface of the second end is predetermined, and whereby when the user locates the reflection of the predetermined object in the reflective device, a quality image of the target facial area may be captured by the image capturing device.

2. The tool of claim 1, wherein the predetermined distance between the first end and the second end corresponds to a minimum focusing distance of the image capturing device.

3. The tool of claim 1, wherein the predetermined distance between the first end and the second end is in the range of about 80 mm to about 600 mm.

4. The tool of claim 1, wherein the target facial area for image capture thereof comprises any one of the following facial areas: cheek area, nose area, forehead area, mouth area, chin area, one or more teeth, periorbital area, entire face area, and combination thereof.

5. The tool of claim 1, wherein the surface area of each of the first end and the second end is in the range of about 1,000 mm2 to about 33,000 mm2.

6. The tool of claim 1, wherein the second end comprises a window for positioning of a lens of the image capturing device when a lens-side surface of the image capturing device is adjacent to the outer surface of the second end.

7. The tool of claim 1, wherein the predetermined location of the reflective device is at a distance of about 3 mm to about 106 mm with respect to a lens of the image capturing device when a lens-side surface of the image capturing device is adjacent to the outer surface of the second end.

8. The tool of claim 1, wherein the reflective surface area of the reflective device is in the range of about 80 mm2 to about 650 mm2.

9. The tool of claim 1, wherein the inner surface of the second end forms an angle with the at least one wall, and the angle ranges from about 80 degrees to about 135 degrees.

10. The tool of claim 1, further comprising a connector for connecting the tool to the image capturing device such that a lens-side surface of the image capturing device is adjacent to the outer surface of the second end.

11. The tool of claim 10, wherein the connector is selected from the group consisting of an adhesive, a hook and loop fastener, a magnetic fastener, a clip-on fastener, an elastic fastener, a suction cup fastener, and combinations thereof.

12. The tool of claim 1, wherein the tool is made from one or more materials selected from the group consisting of paper, paperboard, polystyrene, polystyrene terephthalate, polypropylene, silicon rubber, and combinations thereof.

13. The tool of claim 1, further comprising a light source located at the second end.

14. The tool of claim 1, wherein the image capturing device is part of a mobile device or smart device, or is part of a personal computer, or is a handheld camera.

15. The tool of claim 1, wherein the tool is used in conjunction with a software application having a recognition feature for the predetermined object for enabling automatic image capture by the image capturing device when the user locates the reflection of the predetermined object in the reflective device.

16. The tool of claim 1, wherein the predetermined object is selected from the group consisting of: the user's eye, the user's eye brow, the user's one or more laugh lines, the user's one or more crow's feet in the periorbital area, a reference point in the periorbital area, a reference point in the facial area, or combinations thereof.

17. The tool of claim 1, wherein the tool comprises a carton or a portion of a carton of a product.

18. A method for capturing a quality image using an image capturing device and a tool for use with the image capturing device, the tool comprising a first end and a second end connected by at least one wall, the second end comprising an inner surface facing the first end and an outer surface, and a reflective device located at the inner surface of the second end, the method comprising:
locating the second end of the tool to a lens-side surface of the image capturing device by a connector such that the lens-side surface of the image capturing device is adjacent to the outer surface of the second end;
positioning the first end of the tool over a surface enclosing a target facial area of a user for image capture thereof;
aligning the position of the first end such that the target facial area is enclosed for image capture thereof by enabling the user to locate the reflection of a predetermined object in the reflective device; and
capturing, using the image capturing device, a quality image of the target facial area when the user locates the reflection of the predetermined object in the reflective device,
wherein the distance between the first end and the second end is predetermined and the location of the mirror at the inner surface of the second end is predetermined.

19. The method of claim 18, further comprising using the tool in conjunction with a software application having a recognition feature for the predetermined object for enabling automatic image capture by the image capturing device when the user locates the reflection of the predetermined object in the reflective device.

20. The method of claim 18, wherein the predetermined object is selected from the group consisting of the user's eye, the user's eye brow, the user's one or more laugh lines, the user's one or more crow's feet in the periorbital area, a reference point in the periorbital area a facial area, or combinations thereof.
